(19) United States
(12) Patent Application Publication
(10) Pub. No.: US 2010/0092981 A1
(43) Pub. Date: Apr. 15, 2010

(54) METHODS OF DETECTING HYPERMETHYLATION

(75) Inventor: Anthony P. Shuber, Mendon, MA (US)

Correspondence Address: CHOATE, HALL & STEWART LLP, TWO INTERNATIONAL PLACE, BOSTON, MA, 02110, US

(73) Assignee: Genzyme Corporation, Cambridge, MA (US)

(21) Appl. No.: 12/572,813

(22) Filed: Oct. 2, 2009

Related U.S. Application Data
(62) Division of application No. 11/732,509, filed on Apr. 3, 2007, now abandoned.
(60) Provisional application No. 60/788,994, filed on Apr. 3, 2006.

(51) Int. Cl. C12Q 1/68

(52) U.S. Cl. 435/6

(57) ABSTRACT

Aspects of the invention relate to methods of detecting cancer, such as colon cancer. In aspects of the invention, methods for detecting the presence of hypermethylated genomic DNA in a biological sample are disclosed. Methods of the invention relate to detecting small amounts of hypermethylated nucleic acids in a biological sample.

### RELATED APPLICATION

This application claims priority under 35 U.S.C. §119 (e) from U.S. provisional application Ser. No. 60/788,994, filed Apr. 3, 2006, the content of which is incorporated herein in its entirety.

### FIELD OF THE INVENTION

Aspects of the invention relate to methods of detecting cancer, and colon cancer in particular.

### BACKGROUND OF THE INVENTION

Methylation at certain genetic loci has been associated with cancer.

### SUMMARY OF THE INVENTION

Aspects of the invention relate to methods of detecting cancer (e.g., colon cancer) by detecting hypermethylation at one or more genetic loci. One or more methylation detection assays may be combined with one or more assays for mutation detection, LOH detection, DNA integrity detection (DIA), or detection of one or more other indicia associated with cancer. According to the invention, a detection assay may be performed on a heterogeneous biological sample (e.g., a stool sample, a blood sample, a plasma sample, etc.). According to aspects of the invention, methods may be used to detect low frequency events (e.g., hypermethylation in a relatively small percentage of copies of one or more genetic loci in a biological sample, for example in about 10% or less, about 1% or less, about 0.1% or less, about 0.01% or less, of the copies of one or more genetic loci of interest in a biological sample) indicative of a sub-population of mutant or diseased (e.g., cancerous or precancerous) cells or cellular debris in the biological sample. In some embodiments, armed methylation specific primers (e.g., chimeric primers) may be used to detect subpopulations of hypermethylated nucleic acids. In some embodiments, segmented primers may be used to detect subpopulations of hypermethylated nucleic acids. In some embodiments, a digital analysis may be used to detect subpopulations of hypermethylated nucleic acids (e.g., a hypermethylation analysis may be performed on diluted samples that each contain on average about 1-5, 5-10, or 10-15 individual nucleic acid molecules from the original biological sample (however, these molecules may be amplified in each diluted sample for analysis). In some embodiments, any suitable methylation detection assay may be used. In some embodiments, real-time PCR may be used to quantify levels of hypermethylation in a biological sample. Some aspects of the invention provide threshold levels of hypermethylation indication of the presence of a subpopulation of mutant or diseased cells (e.g., cancer cells, precancer cells, adenoma cells, etc.) in a patient from which the biological sample was obtained.

Aspects of the invention may be used to detect indicia of cancer in any tissue. For example, aspects of the invention may be used to detect indicia of colon, lung, pancreatic, rectal, oral, liver, prostate, kidney, esophageal, nasal, buccal, ovarian, breast, testicular, stomach, gastrointestinal, and/or cerebrospinal, tumor, cancer, adenoma, neoplasia, sarcoma, carcinoma, and/or polyp, etc.

In some embodiments, a nucleic acid may be isolated from a sample using a hybrid capture. A capture nucleic acid complementary to a locus of interest (or complementary to a region near a locus of interest) may be used to isolate target nucleic acid for analysis. A capture nucleic acid may be bound to a solid support. In some embodiments, a capture nucleic acid is bound to a gel material. In some embodiments, a sample is exposed to a capture nucleic acid using chromatography, electrophoresis, and/or any other suitable technique. In some embodiments, a sample may be exposed two or more times (e.g., 3, 4, 5, 6, 7, 8, 9, 10, or more times) to an immobilized capture nucleic acid. In some embodiments, repeated exposure may involve repeated reversed-phase electrophoresis of a sample across an immobilized capture nucleic acid that has a sequence that is complementary to the sequence of a target nucleic acid to be captured. In some embodiments, several different capture nucleic acids may be immobilized and used to capture different target nucleic acids of interest. In some embodiments, one or more immobilized capture nucleic acids may be methylation specific (e.g., include one or more sequences that are complementary to one or more methylated sequences, for example sequences that are specifically complementary to methylated sequences after a nucleic acid sample is exposed to a modifying agent that differentially modifies methylated and unmethylated sequences).

### BRIEF DESCRIPTION OF THE DRAWINGS

FIG. 1 (A) In silico characterisation of RASSF2. Top panel: The RASSF2 gene is located at 20p13, occupies 46 kb of genomic DNA (_strand) and encodes at least three different isoforms, RASSF2A (AY154470), RASSF2B (AY154471) and RASSF2C (AY154472). Bottom panel: All proteins produced by the RASSF2 gene contain predicted RA domains within the C-termini; however, RASSF2B contains a truncated RA domain due to alternative splicing of exons 6, 7, 8, 9 and 10, which introduces a stop codon in exon 10. (B) Amino-acid sequence alignment of human RASSF2A and orthologues in the mouse and rat. Shaded areas represent Ras-association (RA) domains (in dark grey) and coiled-coil SARAH domains (in light grey).

FIG. 2 (a) Frequent methylation of the RASSF2A CpG island is observed in colorectal tumor cell lines as determined by COBRA digest of PCR products. (b) Direct bisulphite sequencing of the RASSF2A CpG island in colorectal tumor cell lines. Each box represents the methylation status of each CG dinucleotide; black box represents a methylated CpG dinucleotide, grey box represents a partially methylated CpG dinucleotide; white box represents an unmethylated CpG dinucleotide; ND represents not determined. (c) Expression of RASSF2A in colorectal tumor cell lines.

FIG. 3 (A) Methylation status of the RASSF2 CpG island in colorectal tumors. Top panel: Methylation of the RASSF2 CpG was tumor-specific. Bottom panel: Methylation was found in 21/30 (70%) primary tumors. M represents methylated-specific PCR; U represents unmethylated-specific PCR; T represents tumor; N represents DNA from corresponding normal mucosa. (B) Cloning and sequencing of the RASSF2 CpG island in colorectal tumor samples. Black box represents a methylated CG dinucleotide; and white box represents an unmethylated CG dinucleotide.

FIG. 4 Methylation status of the RASSF2 CpG island in early adenoma polyps. M represents methylated-specific PCR; U represents unmethylated-specific PCR; P represents polyp; N represents corresponding normal rectal mucosa.

FIG. 5 Gel showing RASSF2A-MSP (MT) mutant.

FIG. 6 Gel showing RASSF2A-USP (WT) wild-type.

### DESCRIPTION OF THE INVENTION

Aspects of the invention relate to methods for detecting the presence of hypermethylated genomic nucleic acid in a biological sample. Methods of the invention are useful for detecting small amounts of hypermethylated nucleic acids (e.g., genomic nucleic acids) in a biological sample that contains primarily non-hypermethylated nucleic acids. According to the invention, a biological sample that contains a small fraction of abnormal cells or cellular debris may contain a small fraction of hypermethylated nucleic acid at a locus where hypermethylation is indicative of disease (e.g., cancer, precancer, adenoma, etc.). The hypermethylated nucleic acid may represent less than 10% (e.g., less than 5%, less than 1%, less than 0.1%, etc.) of the nucleic acid derived from a particular locus in a biological sample.

According to aspects of the invention, one or more genetic loci may be assayed for the presence of hypermethylation. According to aspects of the invention, hypermethylation at one or more of the following loci may be indicative of cancer: Vimentin, RASSF2 (e.g. RASSF2A), and HLTF. However, other loci also may be analyzed.

An assay may include 1, 2, or more (e.g., Vimentin, RASSF2, HLTF, etc., or any combination of two or more there of) methylation markers alone or in combination with one or more other tests (e.g., DIA, mutation detection, cytogenetic analysis, etc.) for nucleic acid abnormalities associated with cancer or precancer.

In some embodiments, aspects of the invention include using one or more segmented primers that are specific for hypermethylated nucleic acid in an amplification reaction. If amplification is detected above a threshold level characteristic of amplification in a control (normal) biological sample, then a subject is identified as having one or more indicia of a disease associated with hypermethylation at the tested locus. The subject may be identified as having cancer, precancer, or adenoma. Alternatively, the subject may be identified as being at risk for cancer, precancer, or adenoma and be tested with one or more follow up tests (e.g., one or more invasive or non-invasive tests).

According to the invention, a segmented primer may include two or more short primers that are complementary to a contiguous genomic sequence. When specifically hybridized, they may be separated by 0, 1, 2, 3 or a few nucleotides on the genomic nucleic acid. Each short primer independently may be between about 5 and about 15 nucleotides long (e.g., about 10, 11, 12, etc. nucleotides long). However, slightly shorter or longer short primers may be used when appropriate. When both (or more) of the short primers of a segmented primer bind to a target sequence, they provide a good substrate for a polymerase that may be used in an amplification reaction. In one embodiment, when only one of the pair of primers in a segmented primer binds, it forms a short hybrid stretch that is not a good substrate for the polymerase. According to the invention, an advantage of using a segmented primer consisting of two or more short primers instead of a longer single primer is better discrimination between a complementary sequence and a sequence that differs by only one or a few bases from the complementary sequence. In one embodiment, the short primers are hybridized under conditions that are useful for discriminating between closely related sequences. Under these conditions, one or both (or more) of the short primers may fail to hybridize (or have significantly reduced hybridization) whereas a longer primer would still hybridize efficiently.

According to aspects of the invention, an amplification (e.g., PCR) reaction for detecting hypermethylated nucleic acid in a sample may include one or more segmented primers (e.g., one or both of the forward and reverse primers for the amplification reaction may be segmented). Any one or more of the short primers used may be specific for hypermethylated nucleic acid (e.g., it binds to methylated nucleic acid and not un-methylated nucleic acid) at a particular locus (e.g., an HLTF, Vimentin, or RASSF2 locus). For example, one or more of the short primers may be designed to overlap with one or more (e.g., 2, 3, 4, 5, 6, 7, 8, 9, 10, or more) CpG dinucleotide sequences at a genetic locus (e.g., a CpG island characteristic of a promoter region). A short primer may be designed so that it only (or preferentially) hybridizes to a nucleic acid with a methylated C in at least one of the CpG dinucleotides. In one embodiment, a methylation specific short primer contains at least one sequence (e.g., nucleotide) that is specific for a methylated base in the genomic nucleic acid (e.g., a methylated C that has been treated with a chemical modification agent that either modifies that methylated C or modifies the un-methylated C). For example, in a modification treatment, a nucleic acid sample may be treated with an agent such as sodium bisulfate. Preferably, the agent modifies unmethylated cytosine to uracil without modifying methylated cytosine. Bisulfite modification treatment is described in U.S. Pat. No. 6,017,704, the entire disclosure of which is incorporated herein by reference. After the modification treatment, an amplification reaction may be performed with at least one segmented primer. The segmented primer may contain one or two (or more) short primers each of which is specific for at least one unmodified CpG dinucleotide sequence or, preferably, for a sequence rich in CpG dinucleotides (a CpG island). Bisulfite treatment converts unmethylated cytosine (C) to uracil (U). Accordingly, in one embodiment, a primer that is specific for a methylated CpG contains a G at the position complementary to a C in the genomic CpG dinucleotide. In one embodiment, both forward and reverse primers are segmented primers with respective template-specific portions that hybridize with unmodified CpG sequences in the target template. The detection of an amplification product indicates methylation at the CpG sequence, preferably, hypermethylation at the CpG island. These results may indicate the presence or onset of a particular disease, e.g., a particular cancer.

Aspects of the invention are useful for detecting the presence of a small amount of hypermethylated nucleic acid in any sample suspected of being heterogeneous and containing rare modified nucleic acids derived from diseased cells. For example, a sample may be a stool sample or a any bodily fluid sample (e.g., serum, plasma, pus, semen, breast-nipple aspirate, urine, saliva, bile, or any other suitable body or organ fluid or secretion).

According to the invention, target nucleic acid may be isolated from a heterogeneous biological sample using a sequence-specific hybrid capture step. The target template can be captured using a capture probe specific for the template. According to the invention, this hybrid capture step is preferably performed prior to CpG modification, and may result in a heterogeneous sample of target nucleic acid that contains both methylated and unmethylated forms of the target nucleic acid.

Aspects of the invention may be performed on diluted samples (e.g., in a digital amplification protocol).

Aspects of the invention may include exposing the hybridized short primers to ligation conditions prior to amplification.

Aspects of the invention may include a 3′ blocked short primer as the upstream primer in a segmented primer pair.

In one aspect, one or more of the segmented primers may include a chimeric primer as the upstream (5′) short primer.

In aspects of the invention, segmented primers may be referred to as tiled primers because they include two or more short primers that “tile” a target region and may serve as a substrate for an enzyme (e.g., thermal polymerase) mediated extension reaction during an amplification reaction.

It should be appreciated that embodiments described in the context of segmented primers may be practiced using typical (non-segmented) primers (e.g., about 10-50 mer oligonucleotides, or shorter or longer oligonucleotides).

The invention may be used to detected rare amounts of any type of hypermethylated nucleic acid (e.g., RNA, mRNA, genomic nucleic acid, etc.) in a biological sample.

In some embodiments, methylation specific amplification may be assayed using real-time PCR (RT-PCR). In certain embodiments, RT-PCR is quantitative PCR (qPCR).

In some embodiments, MSP (methylation-specific primer) amplification may be performed using one or more armed primers.

The disclosure of U.S. Pat. Nos. 5,888,778 and 6,818,404 are incorporated herein in their entirety.

It should be appreciated that any suitable assay may be used to detect methylation levels indicative of cancer at any of the RASSF2A, HLTF, Vimentin, or any other suitable locus, or at any combination of two or more thereof.

It should be appreciated that assays described herein can be used to interrogate one or more loci for long DNA (e.g., DNA integrity), specific cancer-associated mutations, hypermethylation, and/or other chromosomal abnormalities indicative of cancer. If one or more of the assays are positive (e.g., signal above a threshold reference level characteristic of a sample from a normal or healthy subject), then the subject from which the sample was obtained is identified as at risk for, or having cancer or precancer. If all assays are negative, the subject may be identified as healthy or as at low risk for cancer or precancer.

### EXAMPLES

### Example 1

A novel gene, ras association domain family 1 (RASSF1) (reviewed in Agathanggelou et al., 2005) has been identified. The two main isoforms A and C are well expressed in normal tissues; however, expression of RASSF1A but not RASSF1C is lost in a majority of human cancers (Dammann et al., 2000; Lerman and Minna, 2000; Agathanggelou et al., 2001). This loss of expression correlates with hypermethylation of a promoter CpG island. Both A and C isoforms contain a ras association domain in the carboxy terminus, in addition RASSF1A contains a DAGbinding domain at the N-terminus as well as an ATM phosphorylation site consensus sequence. Cells expressing exogenous RASSF1A have been shown to suppress tumorigenicity in nude mice, reduce colony formation and suppress anchorage-independent growth (Dammann et al., 2000; Burbee et al., 2001; Dreijerink et al., 2001). RASSF1 has homology to a known mammalian ras effector nore1 (Vavvas et al., 1998). RASSF1A is brought into the ras signalling pathway by heterodimerization with NORE1 (Ortiz-Vega et al., 2002). RASSF1A is thought to be involved in cell cycle regulation, apoptosis and microtubule stability (Khokhlatchev et al., 2002; Shivakumar et al., 2002; Agathanggelou et al., 2003; Liu et al., 2003; Dallol et al., 2004; Vos et al., 2004). In silico approaches have been used to identify further members of the RASSF family. In a previous report, it was demonstrated that NORE1A like RASSF1A is hypermethylated in a subset of non-small-cell lung carcinomas (NSCLCs). More recently, Irimia et al. (2004) have confirmed these results and have shown that in NSCLC, NORE1A hypermethylation inversely correlates with K-ras mutations. Another member of the RASSF gene family has also been identified and characterized, AD037, located at 10q11.21 (Eckfeld et al., 2004). The AD037 50 CpG island is hypermethylated in lung and breast tumors and this hypermethylation correlates with loss of gene expression. AD037 binds directly to activated Ras in a GTP-dependent manner. Exogenous expression induces ras-dependent apoptosis and inhibits growth of human tumor cell lines. In this example, another member of the RASSF family, RASSF2, has been characterized and it has been demonstrated that a CpG island located in the promoter region of RASSF2A is frequently hypermethylated in colorectal tumors, and the hypermethylation inversely correlates with K-ras mutations in these tumors. While this work was in progress, Vos et al. (2003) independently identified RASSF2 as a novel ras effector. Aspects of the invention relate to detecting epigenetic inactivation of the RASSF2A gene associated with human cancer.

### In Silico Characterization and Cloning of RASSF2

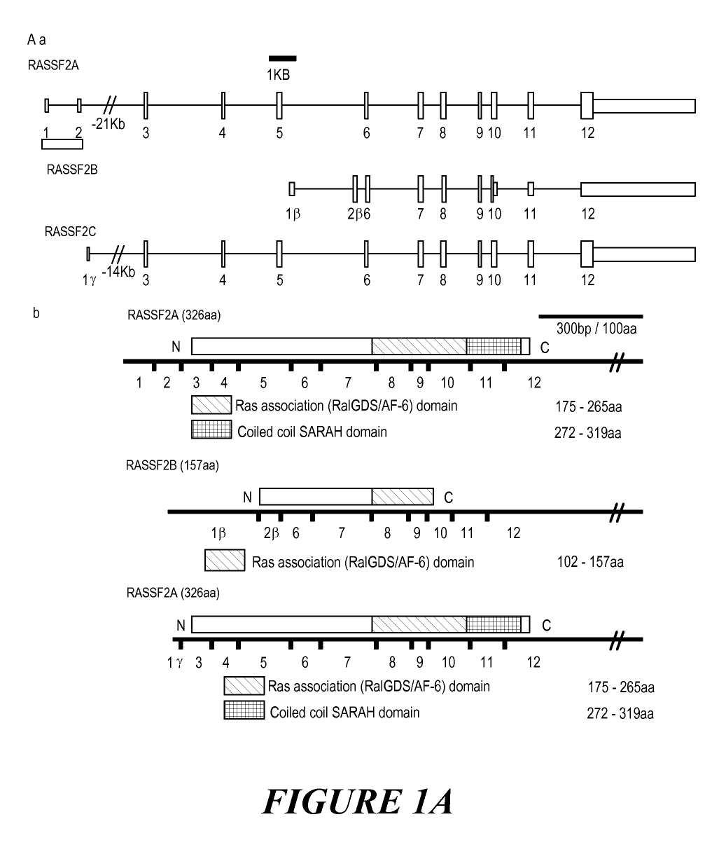

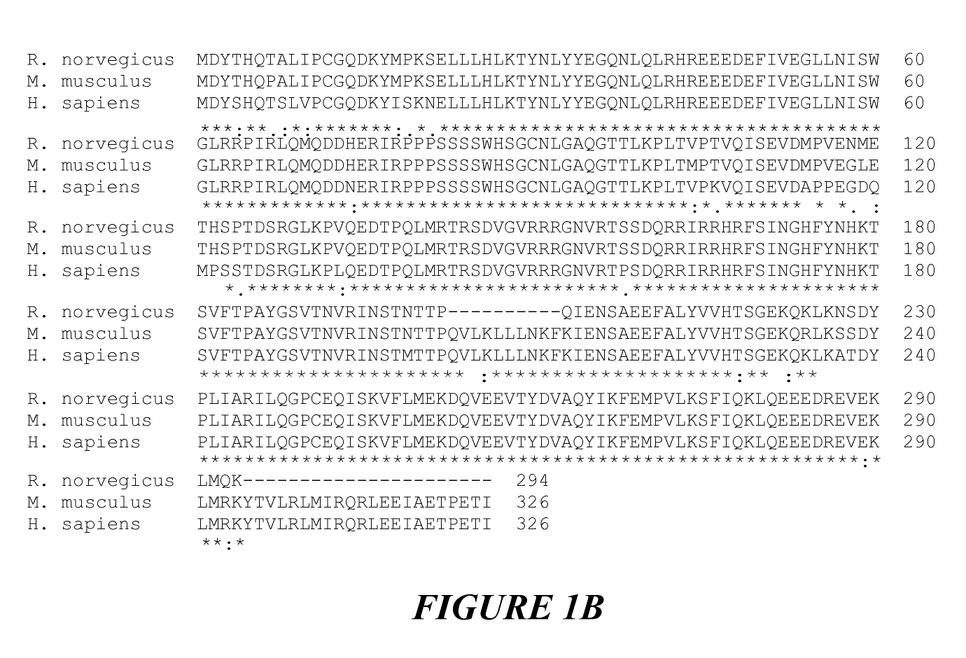

Using bioinformatics analysis, three isoforms of the RASSF2 gene located at 20p13 were identified as RASSF1 homologues (FIG. 1a). RASSF2A, RASSF2B and RASSF2C all contain predicted ras-association (RA) domains, although RASSF2B mRNA produces a much shorter protein with a truncated RA domain. RASSF2A and RASSF2C contain a C-terminal coiled-coil SARAH domain that is absent in RASSF2B. An Itk kinase site at Y224 and a PKCz kinase site at T306 were also predicted for RASSF2A. The first two noncoding exons of RASSF2A are both located within a large CpG island −105 bp- to +1745 by relative to the transcription start site of NM_{—}014737. This 1.8 kb CpG island has an Obs/Exp ratio of 0.97 and a CG percentage of 68.61. RASSF2C and RASSF2B do not have associated CpG islands. Using primers that incorporated the predicted ATG start codon within exon 3 and the predicted TGA stop codon within exon 12, the entire open reading frame of RASSF2A was cloned from a brain-specific cDNA library. Sequencing of the open reading frame confirmed the exonic gene structure seen in FIG. 1a. Comparisons of sequence homology with RASSF1A show that these proteins share 14% amino-acid identity over their entire lengths and 23% identity over their RA domains. The greater degree of homology over the RA domain again indicates functional conservation of this sequence. BLAST database searches of the proteomes of other species identified orthologues in the mouse, rat and cow, all known as Rasst2. The protein identified in Bos Taurus, however, did not encode a predicted RA domain; therefore, it remains to be determined whether this represents a true RASSF2 orthologue. The orthologues identified in mouse and rat, however, were highly similar to human RASSF2A, both sharing 92% amino acid identity over their entire lengths (FIG. 1b).

### RASSF2 CpG Island Methylation Analysis in Colorectal Tumors

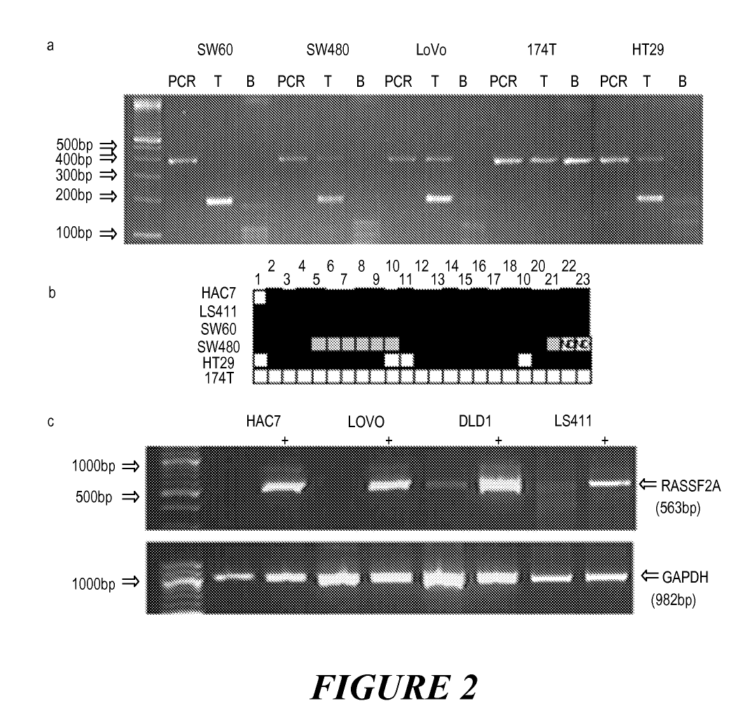

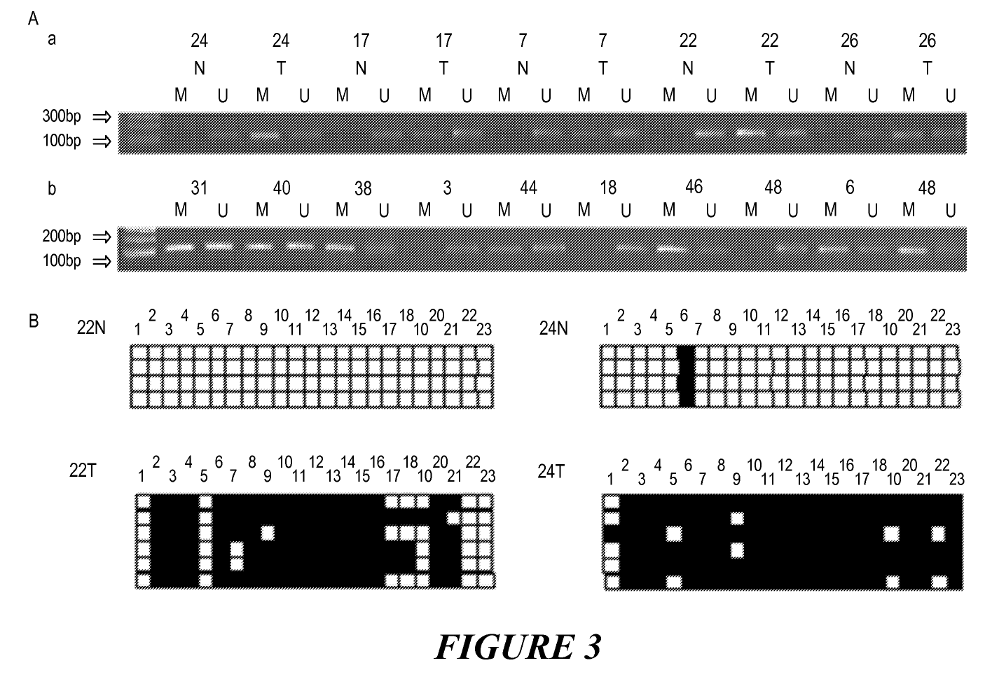

Using combined bisulphite restriction analysis (COBRA) and bisulphite sequencing, the methylation status of the predicted promoter region CpG island was examined in colorectal tumor cell lines. Methylation was observed in 8/9 (89%) colorectal tumor cell lines (FIG. 2a). Direct sequencing showed dense methylation spanning the entire region analyzed (FIG. 2b). RASSF2A CpG island hypermethylation corresponded with loss of RASSF2A expression in colorectal tumor cell lines. Furthermore, treatment of colorectal tumor cell lines with the demethylating agent 5-aza-2-deoxycytidine (5azaDC) reactivated RASSF2A expression (FIG. 2c), showing that promoter hypermethylation is the cause of inactivation of RASSF2A. Next, a methylated-specific PCR (MSP) assay was developed and used to determine whether methylation of the RASSF2A CpG island occurs in colorectal primary tumors (FIG. 3a). Methylation was observed in 21/30 (70%) tumors. Moreover, this methylation was always tumor-specific (FIG. 3a) and was not detected in the matched patient's DNA from normal mucosa (taken >10 cm from the primary). Thus, RASSF2A methylation arose in these patient's tumors as part of, and during, the tumorigenic process. To determine the pattern and extent of methylation, COBRA PCR products from two methylated colorectal tumors and corresponding normal mucosa were cloned and sequenced. FIG. 3b shows that RASSF2A promoter CpG island is hypermethylated in both tumors compared to corresponding normal mucosa samples. No correlation between patient sex, tumor stage, survival or site of tumor was found (for all P>0.05) and RASSF2A methylation; RASSF2A was more frequently methylated in patients of older age (0.025, t-test) (Table 1 shows all the clinicopathological details for the tumors analyzed). Previously, it had been demonstrated that NORE1A was methylated and silenced in a subset of NSCLC (Hesson et al., 2003). In this example, it was found that NORE1A methylation was infrequent in colorectal cancers (CRCs) (⅙ colorectal tumor lines; 3/28 CRC tumors) and there was no association between RASSF2A and NORE1A methylation. While RASSF1A was methylated in 15% ( 5/33) of the colorectal samples, again no association was found between RASSF2A and RASSF1A methylation status. Methylation status of RASSF3 (located at 12q14.1) (Tommasi et al., 2002), another member of the RASSF1 gene family, was also analyzed in colorectal tumor cell lines, and it was found to be unmethylated in all CRC tumor lines (n=8).

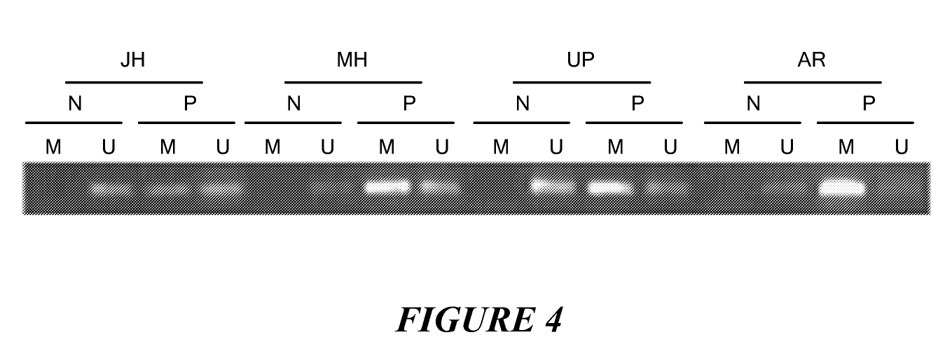

In order to determine the timing of onset of RASSF2A methylation in colon carcinogenesis (Fearon and Vogelstein, 1990), eight colon adenomas were analyzed. RASSF2A promoter CpG island was found to be hypermethylated in seven of eight colon adenomas (FIG. 4), while DNA from matched normal mucosa was unmethylated. Thus, it appears that RASSF2A methylation is an early event in colorectal tumor development. The same colon adenomas were also analyzed for RASSF1A methylation by MSP, and none of the colon adenomas demonstrated hypermethylation of the RASSF1A promoter CpG island.

### K-ras Mutation Status

The colorectal tumor samples were also screened for K-ras mutations at codons 12 and 13. It was found that 75% of colorectal tumors with RASSF2A methylation had wild-type K-ras and this was statistically significant (P=0.048) (Table 1). While this work was in progress, RASSF2 was identified independently by Vos et al. (2003), and they demonstrated direct in vivo binding of RASSF2A to K-ras in a GTP-dependent manner in HEK-293-T cells, whereas only weak association with activated H-Ras was observed. Interaction occurred between the effector domain of K-ras and the RA domain of RASSF2A. Furthermore, exogenous expression of RASSF2A inhibited the growth of lung tumor cells, with enhanced growth inhibition in the presence of activated K-ras (Vos et al., 2003). It seems likely therefore that RASSF2A is a K-ras-preferential effector that promotes growth antagonistic effects in a ras-dependent manner. K-ras is the most commonly constitutively activated ras gene in human cancer (Downward, 2003) and it has been suggested that K-ras may be particularly critical in cancer due to recruitment of specific growth inhibitory downstream effectors. The data described by Vos et al. (2003) suggest that RASSF2A may be one such effector and that its inactivation may be beneficial for tumor cell survival by reducing K-ras apoptotic signals. RASSF2A is thought to interact with MST1 (Khokhlatchev et al., 2002; Praskova et al., 2004); therefore, loss of RASSF2A expression in tumors may result in reduced MST1-mediated proapoptotic signals. A role for RASSF2A in apoptosis is supported by recent data showing growth inhibition of lung tumor cells by increased apoptosis and cell cycle arrest (Vos et al., 2003).

The results in this example show that RASSF2A mRNA expression is lost or drastically downregulated in most colorectal tumor cell lines. Furthermore, expression was fully restored following treatment with a demethylating agent. In colorectal tumor cell lines lacking RASSF2A expression, the entire RASSF2A CpG island was heavily methylated. In colorectal tumors, frequent RASSF2A CpG island methylation was observed in a tumor-specific manner. This was shown using MSP, COBRA and bisulphite sequencing. Furthermore, it has been demonstrated that in a majority of colorectal tumors, RASSF2A methylation occurs in the context of wild-type k-ras. Recently, van Engeland et al. (2002) also reported inverse correlation between RASSF1A methylation and K-ras mutations in colorectal tumors. RASSF2A is more frequently methylated in colorectal tumors compared to RASSF1A or NORE1A methylation (70, 15-45, 11%, respectively) (van Engeland et al., 2002; Wagner et al., 2002; and this report). The results indicate that epigenetic inactivation of RASSF2A, a ras effector, is one of the most frequent events in CRC and may lead to the development of novel therapies affecting the ras signalling pathway, and that further analysis of the protein product of the RASSF2 gene is warranted. Recent yeast two-hybrid results indicate that RASSF2 associates with NORE1, MST1, RASSF3 and several novel proteins that are being investigated (Hesson and Latif, unpublished data). CRC is one of the most frequently occurring cancers in the western world and early detection offers a way to reduce deaths by this cancer. Epigenetic changes in CRC have been widely reported (Suzuki et al. (2002); reviewed in Herman, 2002; Kondo and Issa, 2004). A recent study demonstrated that a subset of genes methylated in colorectal tumors and matching fecal DNA may form a basis for early detection (Muller et al., 2004). RASSF2A methylation is frequent and is tumor-specific in CRCs and may be occurring in early colon tumorigenesis. Hence, RASSF2A methylation may provide a molecular biomarker for early detection of CRC. Ongoing research in may provide further insights into the feasibility of using RASSF2A methylation, with perhaps a combination of other sets of genes, to detect CRC at an early stage.

TABLE 1
Clinicopathological and RASSF2A, NORE1A methylation
and K-RAS mutation status for 30 CRC patients

| | | | Stage | | | | | | |
|---|---|---|---|---|---|---|---|---|---|
| | | | TNM | | | | KRas | RASSF2A | NORE1A |
| No | Age | Sex | Dukes | Site of cancer | Site P/D | 3YRFS | mutation | methylation | methylation |
| 3 | 73 | F | 401 D | Sigmoid | Dist | D | 12/13 | U | U |
| 5 | 78 | M | 410 C | Ascending colon | Prox | NO | 13 | U | U |
| 6 | 62 | F | 300 B | Sigmoid | Dist | NO | — | M | U |
| 7 | 89 | M | 300 B | Mid rectum | Dist | X | — | M | U |
| 13 | 55 | M | 300 B | Rectum | Dist | YES | — | M | U |
| 16 | 61 | F | 400 B | Caecum | Prox | YES | 12 | U | U |
| 17 | 61 | M | 400 B | Sigmoid | Dist | NO | — | M | U |
| 19 | 44 | F | 300 B | Caecum and | Prox | YES | 13 | U | U |
| | | | | descending colon | | | | | |
| 21 | 61 | M | 300 B | Caecum | Prox | YES | — | M | U |
| 22 | 79 | M | 300 B | Sigmoid | Dist | NO | — | M | U |
| 24 | 85 | M | 310 C | Caecum | Prox | NO | 12 | M | U |
| 25 | 76 | F | 410 C | Colon | D? | NO | — | U | U |
| 26 | 76 | F | 410 C | transverse colon | Prox | NO | — | M | M |
| 27 | 48 | M | 301 D | Rectum | Dist | D | 12/13 | U | ND |
| 29 | 74 | F | 400 B | Sigmoid | Dist | YES | — | M | U |
| 30 | 74 | F | 300 B | Ascending colon | Prox | NO | 13 | U | U |
| 31 | 76 | F | 310 C | Transverse colon | Prox | NO | — | M | U |
| 33 | 66 | M | 410 C | Recto-sigmoid | Dist | NO | — | M | U |
| 34 | 71 | F | 301 D | Rectum | Dist | D | — | M | U |
| 36 | 83 | F | 300 B | Ascending colon | Prox | YES | 12 | M | U |
| 38 | 79 | F | 400 B | Caecum | Prox | NO | ND | M | U |
| 40 | 91 | F | 300 B | Ascending colon | Prox | NO | — | M | M |
| 42 | 75 | F | 300 B | Rectum | Dist | NO | 12 | M | U |
| 44 | 80 | F | 400 B | Hepatic flexure | Prox | YES | 12 | M | U |
| 46 | 80 | F | 410 C | Descending colon | Dist | YES | — | M | M |
| 47 | 61 | M | 300 B | Recto-sigmoid | Dist | NO | — | U | ND |
| 48 | 76 | M | 300 B | Rectum | Dist | NO | — | U | U |
| 49 | 82 | F | 400 B | Transverse colon | Prox | NO | 12 | M | U |
| 51 | 61 | M | 400 B | Sigmoid | Dist | YES | — | M | ND |
| 52 | 79 | M | 410 C | Splenic flexure | Dist | NO | — | M | U |

Site P/D; site of tumor is further divided into proximal or distal to the splenic fexure. 3 YRFS; 3-year recurrence-free survival. M; methylated tumor. U; unmethylated tumor. —; no K-ras mutation. ND; not determined

### Tumor Samples

A total of nine colorectal tumor cell lines (SW48, DLD1, 174 T, LS411, LoVo, HAC7, HT29, SW60 and SW480), 33 primary CRCs and eight adenomas were used in this study. For CRCs and adenoma polyps, corresponding normal mucosa and normal rectal mucosa, respectively, were also available.

### Sodium Bisulphite Modification

Sodium Bisulphite modification was performed as described previously (Agathanggelou et al., 2001). Briefly, 0.5-1.0 mg of genomic DNA was denatured in 0.3M NaOH for 15 min at 37° C. Unmethylated cytosine residues were then sulphonated by incubation in 3.12M sodium bisulphite (pH 5.0) (Sigma) and 5 mM hydroquinone (Sigma) in a thermocycler (hybaid Omn-E) for 15 s at 99° C. and 15 min at 50° C. for 20 cycles. Sulphonated DNA was then recovered using the Wizard DNA cleanup system (Promega) according to the manufacturer's instructions. The DNA was desulphonated by addition of 0.3M NaOH for 10 min at room temperature. The converted DNA was then ethanol precipitated and resuspended in 50 ml of water.

### Combined Bisulphite Restriction Analysis (COBRA) and Bisulphite Sequencing

Colorectal tumor cell lines were assayed for RASSF2 CpG island hypermethylation using COBRA assay followed by direct sequencing to confirm methylation status and ascertain the extent of methylation. Seminested PCR (expression analysis and MSP) was performed on a GeneAmp 9700 thermocycler (Perkin-Elmer) and with HotStar Taq DNA polymerase (Qiagen). The primers and conditions used were as follows: initial denaturation for 10 min at 95° C., followed by 30 cycles of 1 min at 94° C., 1 min at 57° C. and 2 min at 74° C. with a final extension for 10 min at 72° C. using the primers RASSF2 F 5′-TTYGAAGAAAGTTGTGGTTTGGAGTTAGTT-3′ and RASSF2 R 5′-TATCCCCAAAACTCTCCRACTTAAAACTA-3′ (where Y=C or T and R=A or G to allow unbiased PCR amplification with regard to methylation status). The reaction volume of 20 ml contained 40 ng bisulphite-modified DNA, 1.5 mM MgCl₂, 0.25 mM dNTPs, 1 mM each primer and 0.5 U HotStar Taq DNA polymerase. A measure of 1 ml of this reaction was then used in a seminested PCR reaction (50 mls) using RASSF2 F (described above) and RASSF2 RN 5′-CCAAACTAAAATCCCAACRACCTCAAA-3′. The same PCR program was used but with 1 U HotStar Taq DNA polymerase and 0.4 mM each primer. This produced a 378 by PCR product. PCR products were then assayed for methylation by incubation with TaqI and BstUI for 2 at 65 and 60° C., respectively, before visualization on a 2% agarose gel with added ethidium bromide. PCR products were also purified using QIAquick PCR purification columns (Qiagen, according to the manufacturer's instructions), reamplified using an ABI BigDye Cycle Sequencing kit V2.0 (PerkinElmer) and analysed using an ABI Prism 3700 DNA sequencer (PerkinElmer). For primary tumors, PCR products were first cloned using the pGEM-T Easy Vector System II (Promega) and transformed into DH5a Escherichia Coli cells before isolation and sequencing of cloned PCR products.

### MSP

The promoter methylation status of RASSF2 in CRCs, adenoma polyps and corresponding normal colorectal epithelium was determined using MSP. Primers were designed to amplify a region within a predicted promoter region at +772 to +979 by relative to the transcription start point of NM_{—}014737 (PromoterInspector at www.genomatix.de) located between the first two noncoding exons of the RASSF2A isoform. The region examined for methylation was also within a CpG island that encompasses the first two noncoding exons of RASSF2A at −105 to +1745 by relative to the transcription start point of NM_{—}014737 (CpGplot at www.ebi.ac.uk). This CpG island is 1.8 kb in length with an ObsExp of 0.97 and a percentage CG of 68.61% (CpGreport at www.ebi.ac.uk). Two sets of primers were used that distinguish between methylated and unmethylated DNA sequences. The conditions and primers were as follows: initial denaturation for 10 min at 95° C., followed by 35 cycles of 30 s at 95° C., 30 s at 58° C. (MSP) or 30 s at 54° C. (USP) and 30 s at 72° C. with a final extension for 10 min at 72° C. using the primers MSP F 5′-GTTCGTCGTCGTTTTTTAGGCG-3′ and MSP R 5′-AAAAACCAACGACCCCCGCG-3′ (for methylated-specific PCR) and USP F 5′-AGTTTGTTGTTGTTTTTTAGGTGG-3′ and USP R 5′-AAAAAACCAACAACCCCCACA-3′ (for unmethylated-specific PCR). The reaction volume of 25 ml contained 100 ng bisulphate-modified DNA, 1.5 mM MgCl₂, 0.25 mM dNTPs, 0.6 mM each primer and 1.25 U HotStar Taq (Qiagen). Products were visualized on a 2% agarose gel with added ethidium bromide.

RASSF1A, NORE1A and RASSF3 methylation analysis was carried out as described previously in Hesson et al. (2004).

Cell Lines and 5azaDC Treatment

Colorectal tumor cell lines were routinely maintained in RPMI 1640 growth media (Invitrogen) supplemented with 10% FCS at 371C, 5% CO2. 5-10×10⁵ cells were plated and allowed 24 h growth before addition of 2.5 mM 5azaDC (Sigma) freshly prepared in ddH20 and filter-sterilized. The medium (including 2.5 mM) was changed every day for 5 days. Controls without 5azaDC were cultured concomitantly in the same manner. RNA was prepared using the RNeasy kit (Qiagen) according to the manufacturer's instructions.

### Reverse Transcriptase-Polymerase Chain Reaction (RT-PCR)

The expression of the RASSF2A isoform was analysed in colorectal tumor cell lines by designing primers specific for exon 2 and exon 7 (RASSF2A). Primer sequences: EXON 2 f 5′-GCGCCTAGAACGTGTTTTTC-3′; EXON 7 R 5′-ACTAGGCGTCCTCACATTGC-3′. In all, 1 mg of cDNA was first created using the SuperScript III cDNA synthesis kit (Invitrogen), 50 ng of cDNA was then analysed by PCR, which consisted of an initial denaturation of 10 min at 95° C. followed by 35 cycles of 95° C. for 30, 58° C. for 30 s, 72° C. for 30 s and a final extension of 10 min at 72° C. The PCR reaction also required 3.0 mM MgCl₂, 0.25 mM dNTPs, 0.8 mM each primer and 1 U HotStar Taq DNA polymerase. This produced a 563 by product for RASSF2A. Primers used for the GAPDH control were 5′-TGAAGGTCGGAGTCAACGGATTTGGT-3′ and 5′-CATGTGGGCCATGAGGTCCACCAC-3′, producing a product of 982 bp. PCR products were visualized on 2% agarose gel with added ethidium bromide.

### K-ras Mutation Analysis

Enriched PCR procedure was carried out using the method of Behn et al. (1998).

### Example 2

The hypermethylation results described in Example 1 for a novel Ras-effector gene, RASSF2A, found 70% ( 21/30) sensitivity in colorectal tumors and 87.5% (⅞) in adenomas. See also Hesson et al., 2005, Oncogene Vol. 24, pp. 3987-3994, the disclosure of which is incorporated herein by reference.

In a further example, 46 diseased colorectal tissues were analyzed (four were repeat tissue samples) for hypermethylation in RASSF2A according to the protocol of Example 1. 100 ng of tissue DNA was bisulfate treated, eluted in a final volume of 45 ul TE and amplified with RASS2FA MSP (MT) and USP (WT) primers. MSP was run in duplicate and USP was run in singlicate.

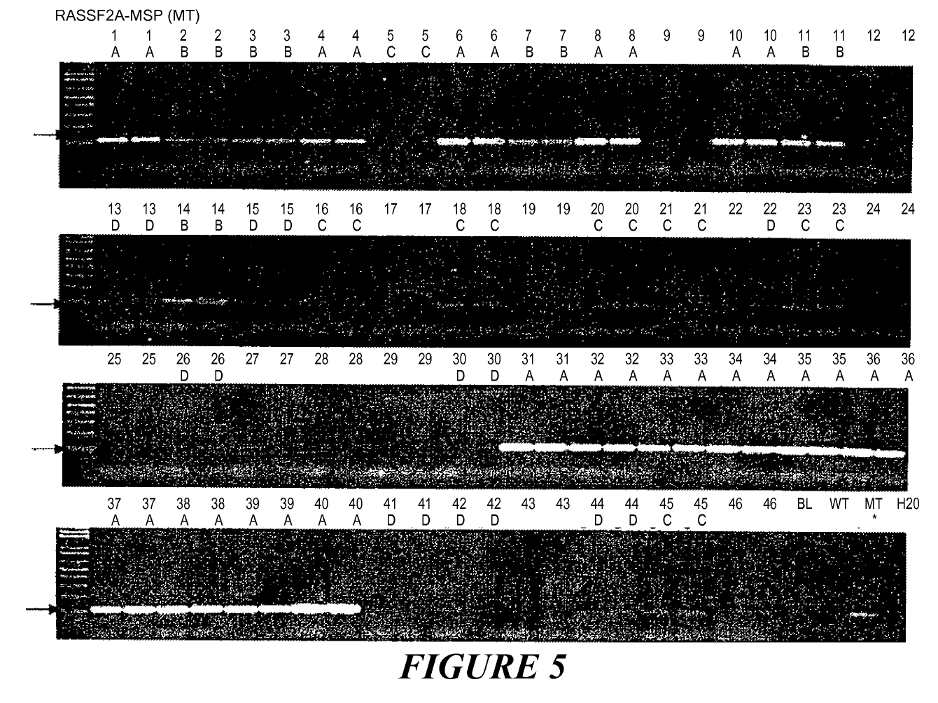

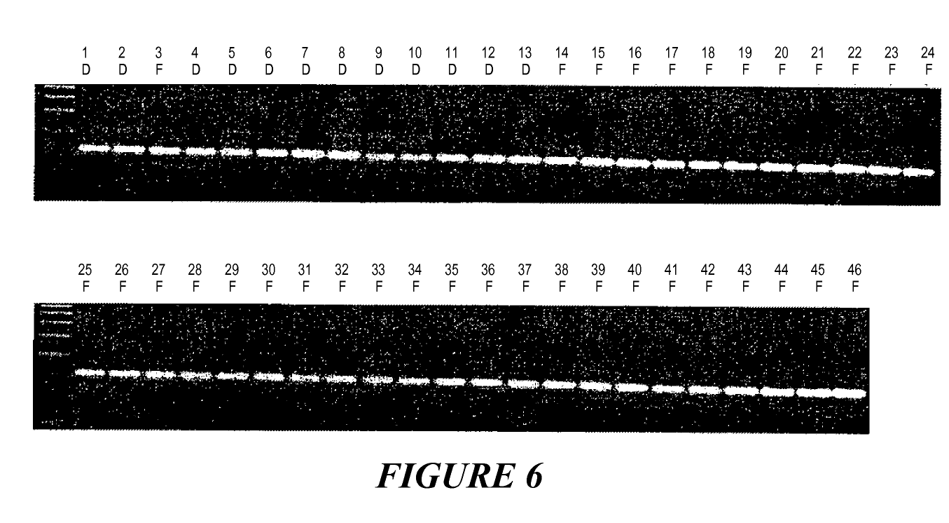

FIGS. 5 and 6 show the results. Sensitivity is shown for all hypermethylation markers. Final sample size was 42 due to 4 repeat tissue samples. RASSF2A sensitivity was calculated using 2 algorithms; Alg 1=band intensity for both replicates ≧D; Alg 2 band intensity ≧C. USP or wild type PCR reactions failed most likely due to strong primer dimer interaction (i.e, USP-F=5′ AGTTTGTTGTTGTTTTTTAGGTGG 3′, USP-R=5′ AAAAAACCAACAACCCCCACA 3′).

TABLE 2

Sensitivity
(N = 42)

| | Alg 1 | Alg 2 |
|---|---|---|
| HLTF | 42.9% | |
| Vimentin | 76.2% | |
| RASSF2A | 81.0% | 64.3% |
| HLTF/V29 | 83.3% | |
| V29/RAS | 90.5% | 83.3% |
| HLTF/V29/RAS | 95.2% | 90.5% |

Results from these tissues were consistent with the tumor tissue results of Example 1 where RASSF2A sensitivity was 64.3-81% based on two algorithms. RASSF2A performs better than HLTF according to both algorithms and performed as well as vimentin for algorithm 1. RASSF2A added to overall methylation informative value by 7.2-11.9%. In some embodiments, the USP wild type primers may be redesigned to improve sensitivity (e.g., by removing sequence characteristics that promote primer dimer formation other secondary structure formation).

### Example 3

### Sample Collection

To avoid any possible effect of the colonoscopic bowel preparation on test results, each subject provided a single stool sample approximately 6-14 days after colonoscopy. In the case of patients with CRC, the sample was provided before beginning the presurgical bowel preparation. Subjects were given detailed instructions and a special stool collection kit that is mounted on the toilet bowl. Immediately after defecation, subjects added 250 mL of a DNA-stabilizing buffer (Olson J, Whitney D H, Durkee K, et al. DNA stabilization is critical for maximizing performance of fecal DNA-based colorectal cancer tests. Diagn Mol Pathol 2005; 14:183-191) to a stool specimen of at least 50 g. Only 10 patients provided less than 50 g of stool, and, of these, 3 subsequently provided an adequate second specimen. The specimen was shipped at room temperature overnight using a coded identifier provided by an external clinical research organization (Carestat Inc., Newton, Mass.) to keep the laboratory blinded to the clinical source. The clinical research organization was responsible for maintaining all of the clinical data files. The collection interval was defined as the number of hours from the time of defecation until the specimen arrived in the laboratory. Stool samples were processed and analyzed without knowledge of clinical information. The details of sample processing and human DNA purification have been described previously.

### Example 4

### DNA Integrity Assay

The DIA was performed using real-time polymerase chain reaction (PCR) as described previously (Whitney D, Skoletsky J, Moore K, et al. Enhanced retrieval of DNA from human fecal samples results in improved performance of colorectal cancer screening test. J Mol Diagn 2004; 6:386-395). The assay was converted to a multiplex format in which 4 primer/probe pairs simultaneously interrogated the presence and quantity of 200-, 1300-, 1800-, and 2400-bp human DNA fragments at 4 loci: 5p21 (locus D), 17p13 (locus E), HRMTILI (locus X), and LOC91199 (locus Y).

### Example 5

### Methylation Assay for HLTF and Vimentin

Stool samples were processed for vimentin and Helicaselike Transcription Factor (HLTF) analysis according to Whitney et al (Whitney D, Skoletsky J, Moore K, et al. Enhanced retrieval of DNA from human fecal samples results in improved performance of colorectal cancer screening test. J Mol Diagn 2004; 6:386-395) by using the following capture sequences: vimentin (Vimcp50a: 5′-GGCCAGCGAGAAGTCCACCGAGTCCTGCAGGAGCCGC-3′; Vimcp29b: 5′-GAGCGAGAGTGGCAGAGGACTGGACCCCGCCGAGG-3′), and HLTF (methylation-specific polymerase chain reaction [MSP]5 cp-. 5′-CAAATGAACCTGACCTTCCCGGCGTTCCTCTGCGTTC-3′). Bisulfite conversion of DNA was performed as previously described (Herman J G, Graff Jr, Myohanen S. et al. Methylation-specific PCR: A novel PCR assay for methylation status of CpG islands. Proc Natl Acad Sci USA 1996; 93:9821-9826; Toyota M, Ahuja N, Ohe-Toyota M, et al. CpG island methylator phenotype in colorectal cancer. Proc Natl Acad Sci USA 1999; 96:8681-8686). MSP PCR reactions were performed using 0.5-μmol/L armed primers for either HLTF MSP-5 or vimentin MSP-29 (IDT, Coralville, Iowa). HLTF MSP-5 primer sequences have been reported previously (forward unmethylated 5′-AAACAACATCAACATCTAACTAAACTCACA-3′; reverse unmethylated 5′-GGGGATGTTTTTAGGTTGTTAGATTGAGT-3′; forward methylated 5′-ACGTCGACGTCTAACTAAACTCGCGA-3′; reverse methylated 5′-GACGTTTTTAGGTCGTTAGATCGAGC-3′; Kim Y H, Petko Z, Dzieciatkowski S, et al. CpG island methylation of genes accumulates during the adenoma progression step of the multistep pathogenesis of colorectal cancer. Genes Chromosomes Cancer 2006; 45:781-789). Modified HLTF MSP-5 methylation-specific forward primers 5′-GACGTCTAACTAAACTCGCGA-3′ and reverse primers 5′-TAGGTCGTTAGATCGAGC-3′ were extended by a 5′ tag sequence 5′-GCGGTCCCAATAGGGTCAGT-3′, which is not derived from the HLTF sequence, but which allows for more robust sequence-specific template amplification. Vimentin MSP-29 primer sequences have been reported previously (forward transcript amplification primer 5′-CACGAAGAGGAAATCCGGAGC-3′; reverse transcript amplification primer 5′-CAGGGCGTCATTGTTCCG-3′; forward MS-PCR primer 5′-TCGTTTCGAGGTTTTCGCGTTAGAGAC-3′; and reverse MS-PCR primer 5′-CGACTAAAACTCGACCGACTCGCGA-3′; Chen W D, Han Z J, Skoletsky J, et al. Detection in fecal DNA of colon cancer-specific methylation of the nonexpressed vimentin gene. J Natl Cancer Inst 2005; 97:1124-1132). In some embodiments, both forward and reverse primers are extended by the addition of a tag sequence, e.g., a 5′-GCGGTCCC-3′ at the 5′ end. Primers were combined with 1× HotStar buffer, 1.25 U HotStar polymerase (Qiagen, Alameda, Calif.), 200 μmol/L deoxynucleoside triphosphate (Promega), and 10 μL (capture stool) DNA in a final volume of 50 μL. Cycling conditions were 95° C. for 14.5 minutes followed by 40 cycles of 94° C. for 30 seconds, 57° C. (HLTF), 68° C. (vimentin methylated) or 62° C. (vimentin unmethylated) for 1 minute, 72° C. for 1 minute, with final 72° C. for 5 minutes. Samples were visualized on 4% NuSieve 3:1 agarose (FMC, Rockland, Me.) gels using a Stratagene EagleEye II (Stratagene, La Jolla, Calif.) still-image system. Samples were scored as positive if the PCR band intensity exceeded a previously determined level. Positive samples were repeated in duplicate to confirm methylation status.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

I claim:

1-7. (canceled)

8. A method for detecting hypermethylated nucleic acids, the method comprising steps of:
(a) providing a heterogeneous biological sample;
(b) providing one or more segmented primers specific for the hypermethylated nucleic acids, each segmented primer comprising two or more short primers, wherein none of said short primers alone is capable of serving as an amplification primer, but said two or more short primers hybridized to said hypermethylated nucleic acids adjacent to each other serve as a primer for amplification;
(c) performing an amplification reaction using the one or more segmented primers; and
(d) detecting amplification product; wherein the presence of amplification product is indicative of the presence of hypermethylated nucleic acids in the sample.

9. The method of claim 8, wherein each of the two or more short primers has a length between 5 and 15 nucleotides long.

10. The method of claim 9, wherein the two or more short primers, when hybridized to the hypermethylated nucleic acids, are separated by no more than 3 nucleotides from each other.

11. The method of claim 8, wherein at least one of the short primers binds to methylated nucleic acid but not unmethylated nucleic acids.

12. The method of claim 8, wherein the hypermethylated nucleic acids comprise methylated cytosine (C) at one or more CpG (cytosine-guanosine) sites.

13. The method of claim 12, wherein the one or more CpG sites are located in a CpG island.

14. The method of claim 12, wherein the one or more CpG sites are located in a promoter region.

15. The method of claim 12, wherein at least one of the short primers comprises a sequence that overlaps with the one or more CpG sites.

16. The method of claim 15, wherein the at least one of the short primers preferentially hybridizes to the one or more CpG sites with methylated C.

17. The method of claim 16, wherein the at least one of the short primers contains a G at the position complementary to a methylated C in the one or more CpG sites.

18. The method of claim 16, wherein the method further comprises a step of treating the biological sample with an agent that modifies unmethylated C before step (b) such that the unmethylated C is converted to uracil (U).

19. The method of claim 8, wherein the one or more segmented primers are used as both forward and reverse primers in the amplification reaction.

20. The method of claim 8, wherein the amplification product is detected relative to a threshold level.

21. A method of detecting a disease associated with hypermethylation at a target locus, the method comprising steps of:
(a) providing a heterogeneous biological sample obtained from an individual;
(b) contacting the heterogeneous biological sample with one or more segmented primers that specifically bind to a hypermethylated nucleic acid sequence at the target locus, each segmented primer comprising two or more short primers, wherein none of said short primers alone is capable of serving as an amplification primer, but said two or more short primers hybridized to said hypermethylated nucleic acid sequence adjacent to each other serve as a primer for amplification;
(c) performing an amplification reaction using the one or more segmented primers; and
(d) detecting amplification relative to a threshold level; wherein the detection of amplification above the threshold level indicates that the individual has or is at risk of the disease associated with hypermethylation at the target locus.

22. The method of claim 21, wherein each of the two or more short primers has a length between 5 and 15 nucleotides long.

23. The method of claim 21, wherein the threshold level corresponds to level of amplification in a control sample from a normal or healthy individual.

24. The method of claim 21, wherein less than 1% of the nucleic acids in the heterogeneous biological sample are hypermethylated at the target locus.

25. The method of claim 21, wherein less than 0.1% of the nucleic acids in the heterogeneous biological sample are hypermethylated at the target locus.

26. The method of claim 21, wherein the heterogeneous biological sample is obtained from a bodily fluid.

27. The method of claim 26, wherein the bodily fluid is selected from the group consisting of serum, plasma, pus, semen, breast-nipple aspirate, urine, saliva, and bile.

28. The method of claim 21, wherein the heterogeneous biological sample is obtained from stool.

29. The method of claim 21, further comprising a step of isolating hypermethylated nucleic acids from the heterogeneous biological sample using sequence-specific hybrid capture.

30. The method of claim 21, wherein the amplification is performed in a diluted biological sample.

31. The method of claim 30, wherein the diluted biological sample contains on average about 10 to 15 individual nucleic acid molecules from the original biological sample.

32. The method of claim 30, wherein the diluted biological sample contains on average about 5 to 10 individual nucleic acid molecules from the original biological sample.

33. The method of claim 30, wherein the diluted biological sample contains on average about 1 to 5 individual nucleic acid molecules from the original biological sample.

34. The method of claim 30, wherein the amplification is detected by digital analysis.

35. The method of claim 21, wherein the disease is cancer, precancer or adenoma.

36. The method of claim 21, further comprising performing an assay selected from the group consisting of DNA integrity assays, mutation detection, and cytogenetic analysis.


SEQUENCE LISTING (30 sequences)

SEQ ID NO: 1 - DNA, 30 bp, Artificial Sequence
  Description of Artificial Sequence Synthetic primer
ttygaagaaagttgtggtttggagttagtt

SEQ ID NO: 2 - DNA, 29 bp, Artificial Sequence
  Description of Artificial Sequence Synthetic primer
tatccccaaaactctccracttaaaacta

SEQ ID NO: 3 - DNA, 27 bp, Artificial Sequence
  Description of Artificial Sequence Synthetic primer
ccaaactaaaatcccaacracctcaaa

SEQ ID NO: 4 - DNA, 22 bp, Artificial Sequence
  Description of Artificial Sequence Synthetic primer
gttcgtcgtcgttttttaggcg

SEQ ID NO: 5 - DNA, 20 bp, Artificial Sequence
  Description of Artificial Sequence Synthetic primer
aaaaaccaacgacccccgcg

SEQ ID NO: 6 - DNA, 24 bp, Artificial Sequence
  Description of Artificial Sequence Synthetic primer
agtttgttgttgttttttaggtgg

SEQ ID NO: 7 - DNA, 21 bp, Artificial Sequence
  Description of Artificial Sequence Synthetic primer
aaaaaaccaacaacccccaca

SEQ ID NO: 8 - DNA, 20 bp, Artificial Sequence
  Description of Artificial Sequence Synthetic primer
gcgcctagaacgtgtttttc

SEQ ID NO: 9 - DNA, 20 bp, Artificial Sequence
  Description of Artificial Sequence Synthetic primer
actaggcgtcctcacattgc

SEQ ID NO: 10 - DNA, 26 bp, Artificial Sequence
  Description of Artificial Sequence Synthetic primer
tgaaggtcggagtcaacggatttggt

SEQ ID NO: 11 - DNA, 24 bp, Artificial Sequence
  Description of Artificial Sequence Synthetic primer
catgtgggccatgaggtccaccac

SEQ ID NO: 12 - DNA, 24 bp, Artificial Sequence
  Description of Artificial Sequence Synthetic primer
agtttgttgttgttttttaggtgg

SEQ ID NO: 13 - DNA, 21 bp, Artificial Sequence
  Description of Artificial Sequence Synthetic primer
aaaaaaccaacaacccccaca

SEQ ID NO: 14 - DNA, 37 bp, Artificial Sequence
  Description of Artificial Sequence Synthetic oligonucleotide
ggccagcgagaagtccaccgagtcctgcaggagccgc

SEQ ID NO: 15 - DNA, 35 bp, Artificial Sequence
  Description of Artificial Sequence Synthetic oligonucleotide
gagcgagagtggcagaggactggaccccgccgagg

SEQ ID NO: 16 - DNA, 37 bp, Artificial Sequence
  Description of Artificial Sequence Synthetic oligonucleotide
caaatgaacctgaccttcccggcgttcctctgcgttc

SEQ ID NO: 17 - DNA, 30 bp, Artificial Sequence
  Description of Artificial Sequence Synthetic primer
aaacaacatcaacatctaactaaactcaca

SEQ ID NO: 18 - DNA, 29 bp, Artificial Sequence
  Description of Artificial Sequence Synthetic primer
ggggatgtttttaggttgttagattgagt

SEQ ID NO: 19 - DNA, 26 bp, Artificial Sequence
  Description of Artificial Sequence Synthetic primer
acgtcgacgtctaactaaactcgcga

SEQ ID NO: 20 - DNA, 26 bp, Artificial Sequence
  Description of Artificial Sequence Synthetic primer
gacgtttttaggtcgttagatcgagc

SEQ ID NO: 21 - DNA, 21 bp, Artificial Sequence
  Description of Artificial Sequence Synthetic primer
gacgtctaactaaactcgcga

SEQ ID NO: 22 - DNA, 18 bp, Artificial Sequence
  Description of Artificial Sequence Synthetic primer
taggtcgttagatcgagc

SEQ ID NO: 23 - DNA, 20 bp, Artificial Sequence
  Description of Artificial Sequence Synthetic oligonucleotide
gcggtcccaatagggtcagt

SEQ ID NO: 24 - DNA, 21 bp, Artificial Sequence
  Description of Artificial Sequence Synthetic primer
cacgaagaggaaatccggagc

SEQ ID NO: 25 - DNA, 18 bp, Artificial Sequence
  Description of Artificial Sequence Synthetic primer
cagggcgtcattgttccg

SEQ ID NO: 26 - DNA, 27 bp, Artificial Sequence
  Description of Artificial Sequence Synthetic primer
tcgtttcgaggttttcgcgttagagac

SEQ ID NO: 27 - DNA, 25 bp, Artificial Sequence
  Description of Artificial Sequence Synthetic primer
cgactaaaactcgaccgactcgcga

SEQ ID NO: 28 - protein, 294 aa, Rattus norvegicus
MDYTHQTALIPCGQDKYMPKSELLLHLKTYNLYYEGQNLQLRHREEEDEFIVEGLLNISWGLRRPIRLQMQDDHERIRPPPSSSSWHSGCNLGAQGTTLKPLTVPTVQISEVDMPVENMETHSPTDSRGLKPVQEDTPQLMRTRSDVGVRRRGNVRTSSDQRRIRRHRFSINGHFYNHKTSVFTPAYGSVTNVRINSTMTTPQIENSAEEFALYVVHTSGEKQKLKNSDYPLIARILQGPCEQISKVFLMEKDQVEEVTYDVAQYIKFEMPVLKSFIQKLQEEEDREVEKLMQK

SEQ ID NO: 29 - protein, 326 aa, Mus musculus
MDYTHQPALIPCGQDKYMPKSELLLHLKTYNLYYEGQNLQLRHREEEDEFIVEGLLNISWGLRRPIRLQMQDDHERIRPPPSSSSWHSGCNLGAQGTTLKPLTMPTVQISEVDMPVEGLETHSPTDSRGLKPVQEDTPQLMRTRSDVGVRRRGNVRTSSDQRRIRRHRFSINGHFYNHKTSVFTPAYGSVTNVRINSTMTTPQVLKLLLNKFKIENSAEEFALYVVHTSGEKQRLKSSDYPLIARILQGPCEQISKVFLMEKDQVEEVTYDVAQYIKFEMPVLKSFIQKLQEEEDREVEKLMRKYTVLRLMIRQRLEEIAETPETI

SEQ ID NO: 30 - protein, 326 aa, Homo sapiens
MDYSHQTSLVPCGQDKYISKNELLLHLKTYNLYYEGQNLQLRHREEEDEFIVEGLLNISWGLRRPIRLQMQDDNERIRPPPSSSSWHSGCNLGAQGTTLKPLTVPKVQISEVDAPPEGDQMPSSTDSRGLKPLQEDTPQLMRTRSDVGVRRRGNVRTPSDQRRIRRHRFSINGHFYNHKTSVFTPAYGSVTNVRINSTMTTPQVLKLLLNKFKIENSAEEFALYVVHTSGEKQKLKATDYPLIARILQGPCEQISKVFLMEKDQVEEVTYDVAQYIKFEMPVLKSFIQKLQEEEDREVEKLMRKYTVLRLMIRQRLEEIAETPATI